(19) United States
(12) Patent Application Publication
(10) Pub. No.: US 2009/0282908 A1
(43) Pub. Date: Nov. 19, 2009

(54) ELECTRIFIABLE FABRIC

(75) Inventors: Habib Homayoun, Beaverton, OR (US); Jack Coy, Wapwallopen, PA (US); Shahid Bhatti, Hatfield, PA (US); Melvin Campf, Lake Oswego, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: Thermogear, Inc., Lake Oswego, OR (US)

(21) Appl. No.: 12/463,992

(22) Filed: May 11, 2009

Related U.S. Application Data
(60) Provisional application No. 61/052,099, filed on May 9, 2008.

(51) Int. Cl. G01N 33/00; D03D 15/00

(52) U.S. Cl. 73/159; 442/301; 442/189

(57) ABSTRACT

Embodiments of the present invention provide an electrifiable fabric made up of conductive fibers and non-conductive fibers. Garments may be created with the electrifiable fabric. Electricity may be applied to the electrifiable fabric, where the ratio of non-conductive fibers to conductive fibers provides an electrical resistance to thereby generate heat.

### CROSS REFERENCE TO RELATED APPLICATIONS

The present application claims priority to U.S. patent application Ser. No. 61/052,099 filed May 9, 2008, entitled “ELECTRIFIABLE FABRIC” the entire specification of which is hereby incorporated by reference its entirety for all purposes, except for those sections, if any, that are inconsistent with this specification.

### TECHNICAL FIELD

Embodiments of the present invention relate to the field of fabrics and their use, and more particularly, to an electrifiable fabric that is made up of conductive fibers and non-conductive fibers.

### BACKGROUND

Garments are worn for many purposes including keeping the person who is wearing the garment warm. Often when a garment provides improved warmth, the garment is bulkier and heavier making the garment more cumbersome.

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIG. 1 illustrates an example of portion of an electrifiable fabric, in accordance with various embodiments of the present invention; and

FIG. 2 illustrates an example of a vest made with electrifiable fabric of FIG. 1, in accordance with various embodiments of the present invention.

### DETAILED DESCRIPTION OF EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the present invention, the phrase “A/B” means A or B. For the purposes of the present invention, the phrase “A and/or B” means “(A), (B), or (A and B)”. For the purposes of the present invention, the phrase “at least one of A, B, and C” means “(A), (B), (C), (A and B), (A and C), (B and C), or (A, B and C)”. For the purposes of the present invention, the phrase “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,”0 “having,” and the like, as used with respect to embodiments of the present invention, are synonymous.

Embodiments of the present invention provide an electrifiable fabric that is made up of conductive fibers and non-conductive fibers, and garments that are made up of such an electrifiable fabric.

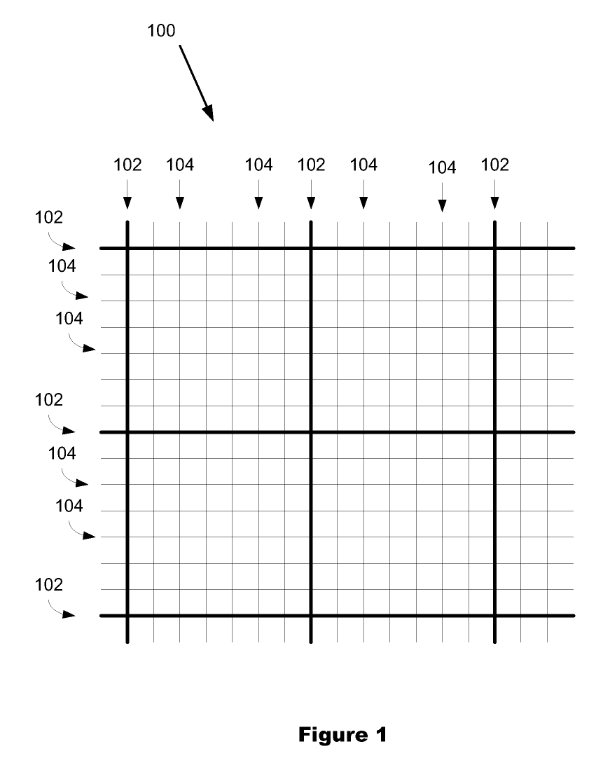

FIG. 1 illustrates an exemplary electrifiable fabric 100, in accordance with various embodiments of the present invention. The fabric 100 includes conductive fibers 102 interspersed with non-conductive fibers 104, which may be arranged in grid-like pattern. An example of conductive fiber 102 includes X-STATIC®, available from Noble Biomaterials, Inc. of Scranton, Pa. An example of non-conductive fiber includes nylon fiber, which is well known in the art. As used herein, fiber refers to thread, yarn and other similar terms known in the art.

In accordance with various embodiments of the present invention, a ratio of non-conductive fibers 104 to conductive fibers 102 may be in a range between 2 to 1 and 130 to 1. In accordance with various exemplary embodiments, the conductive fabric 100 may have a ratio between 6 to 1 and 8 to 1 of non-conductive fibers 104 to conductive fibers 102. As may be seen in FIG. 1, in accordance with an exemplary embodiment, the conductive fabric 100 may have a ratio of 7 to 1 non-conductive fibers 104 to conductive fibers 102. In accordance with various embodiments, conductive fabric 100 may include 100% conductive fibers 102, i.e., no non-conductive fibers 104 are included. In accordance with various embodiments of the present invention, there may be different ratios of non-conductive threads to conductive threads in the vertical direction and the horizontal direction. For example, the ratio in the vertical direction may be 8 to 1 while the ratio in the horizontal direction may be 3 to 1.

The ratio of non-conductive fibers 104 to conductive fibers 102 creates an electrical resistance within fabric 100. For example, a 7 to 1 ratio provides approximately 2.5 ohms per square inch.

In accordance with various embodiments, the fabric 100 is created by a weaving process that involves weaving the conductive fibers 102 and non-conductive fibers 104 together. Those skilled in the art will understand that other techniques may be used to create the fabric such as, for example, knitting, braiding and twill-type weaving.

In accordance with various embodiments, the fabric may have a fiber count in a range of 70×70 to 130×130 per square inch, with an exemplary embodiment having a fiber count of 100×100 per square inch. The size of the fibers, both conductive 102 and non-conductive 104, may be in a range of 40-125 deniers, in accordance with various embodiments. In accordance with an exemplary embodiment, the fibers 102, 104 have a size of 70 deniers.

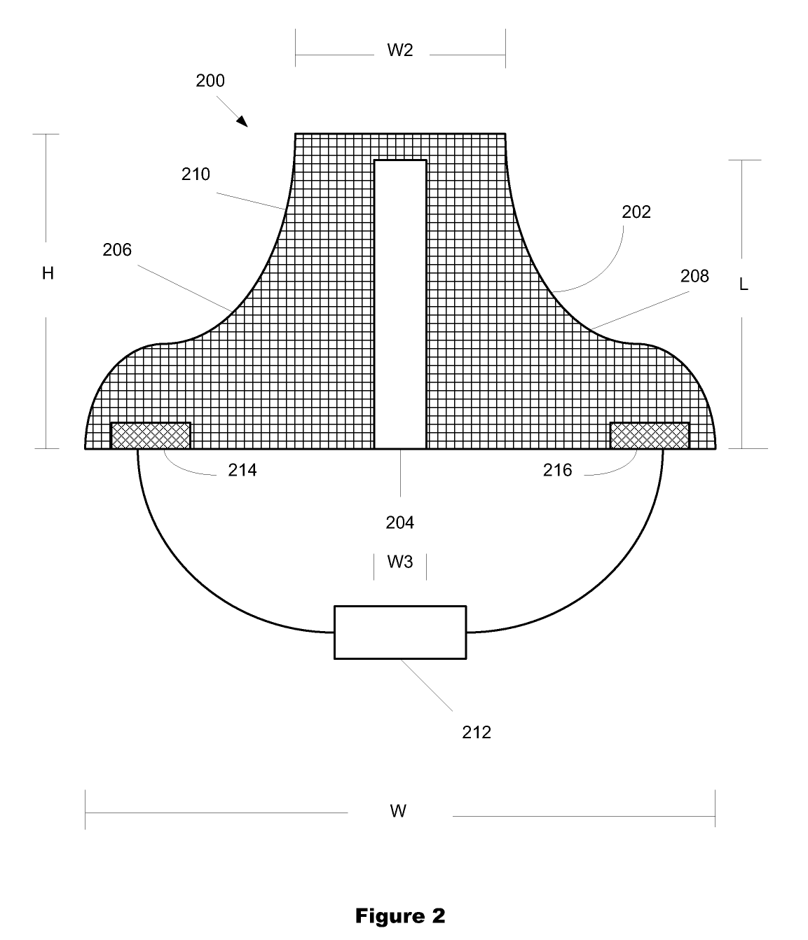

FIG. 2 illustrates an example of a vest 200 made at least partially with electrifiable fabric 100. The vest may include an outer layer (not shown) and/or an inner layer (not shown) to which a layer 202 of electrifiable fabric 100 may be coupled. The outer layer may be made from various fabrics that may facilitate the prevention of loss of heat and may be waterproof. The inner layer may be located on an opposite side of the layer 202 from the outer layer. The inner layer may be made from various fabrics that may also be waterproof. In accordance with various embodiments, the inner and outer layers may have the same shape as the layer 202 of electrifiable fabric 100, or the inner and/or outer layers may have a shape that is different than layer 202. Furthermore, in accordance with various embodiments, there may simply be a single layer that covers the entire layer 202.

In accordance with various embodiments, the layer 202 of electrifiable fabric 100 may include a cut in the form of divider 204 that allows the fibers 102, 104 to extend across the vest from side 206 to side 208 at a more consistent length. In accordance with various embodiments, the layers may be sewn together at their sides and layer 202 may also be sewn to the inner layer and/or outer layer at the divider 204.

Exemplary measurements for the vest 200 include an overall width W of approximately 34 inches and an overall height H of approximately 16 inches, with the width W2 of top portion 210 of the vest 200 being approximately 10 inches. The divider 204 may have exemplary dimensions of a length L of approximately 13 inches and a width W3 of approximately 1.5 inches.

As may be seen in FIG. 2, in accordance with various embodiments, a power source 212 may be coupled to the vest 200 via, for example, braided bus bars 214, 216. Other examples of bus bars include twisted wire pair bus bars, and other types of bus bars generally made from conductive wires. The power source 212 may be in the form of, for example, a battery supply or a light or solar power supply. In accordance with various embodiments, the power source provides approximately 8 volts of voltage to the electrifiable fabric 100 of the vest 200. This causes current to flow through the conductive fibers 102. Because of the resistance provided by the combination of the conductive fibers 102 and non-conductive fibers 104, heat is generated that thereby causes the vest 200 to get warm.

While a vest 200 has been illustrated in FIG. 2, it should be readily apparent to those skilled in the art that other garments, for example, coats, shirts, socks, boot inserts, pants, blankets, wraps, etc., may be created with electrifiable fabric in accordance with various embodiments of the present invention. Additionally, garments may be created with electrifiable fabric 100 having variable and/or different resistance portions. For example, the ratio of non-conductive fibers 104 to conductive fibers 102 may be varied within fabric 100 or different pieces of fabric 100 having different ratios may be coupled together, as well as having different shapes, which also affects the resistance. Multiple bus bars of differing sizes may be coupled to the garment to allow for differing amounts of power to be applied to the garment.

In accordance with various embodiments of the present invention, a method of determining a ratio of non-conductive fibers 104 to conductive fibers 102 within at least a portion of fabric 100 may include determining an amount of heat desired within the portion of the fabric 100. In order to achieve the desired amount of heat within the portion of the fabric 100, an amount of desired resistance within the portion of the fabric 100 may be determined. This may be done, for example, by calculating the area of the portion of the fabric. Based upon the determined desired amount of resistance, a ratio of non-conductive fibers 104 to conductive fibers 102 within the portion of fabric 100 may be determined. The method may also include determining a thread count of fibers within the portion of the fabric 100. This process may be repeated for one or more other portions of the fabric 100 if desired, or the entire fabric 100 may have the same ratio of non-conductive fibers 104 to conductive fibers 102 and the same thread count, if desired.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments illustrated and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. An electrifiable fabric comprising non-conductive fibers and conductive fibers, wherein the ratio of non-conductive fibers to fibers is in a range between 2 to 1 and 130 to 1.

2. The electrifiable fabric of claim 1, wherein the ratio is between 6 to 1 and 8 to 1.

3. The electrifiable fabric of claim 2, wherein the ratio is 7 to 1.

4. The electrifiable fabric of claim 1, wherein the non-conductive fibers and conductive fibers have a size in a range of 40 to 125 deniers.

5. The electrifiable fabric of claim 4, wherein the non-conductive fibers and conductive fibers have a size 70 deniers.

6. The electrifiable fabric of claim 1, wherein the fabric has a thread count in a range of 70×70 to 130×130 per square inch.

7. The electrifiable fabric of claim 6, wherein the fabric has a thread count of 100×100 per square inch.

8. A method comprising:
determining an amount of heat desired within at least a portion of fabric;
determining an amount of desired resistance with the at least a portion of fabric based upon the determined amount of heat desired; and
determining a ratio of non-conductive fibers to conductive fibers within the at least a portion of fabric based upon the determined amount of desired resistance.

9. The method of claim 8, further comprising:
determining a thread count of fibers within the at least a portion of fabric.

10. The method of claim 8, further comprising:
determining an amount of heat desired within another portion of the fabric;
determining an amount of desired resistance with the another portion of the fabric based upon the determined amount of heat desired; and
determining a ratio of non-conductive fibers to conductive fibers within the another portion of the fabric based upon the determined amount of desired resistance.

11. The method of claim 10, further comprising:
determining a thread count of fibers within the another portion of the fabric.

12. A garment comprising:
an electrifiable fabric comprising non-conductive fibers and conductive fibers, wherein the ratio of non-conductive fibers to fibers is in a range between 2 to 1 and 130 to 1.

13. The garment of claim 12, wherein the ratio is between 6 to 1 and 8 to 1.

14. The garment of claim 13, wherein the ratio is 7 to 1.

15. The garment of claim 12, wherein the non-conductive fibers and conductive fibers have a size in a range of 40 to 125 deniers.

16. The garment of claim 15, wherein the non-conductive fibers and conductive fibers have a size of 70 deniers.

17. The garment of claim 12, wherein the fabric has a thread count in a range of 70×70 to 130×130 per square inch.

18. The garment of claim 17, wherein the fabric has a thread count of 100×100 per square inch.

19. The garment of claim 12, further comprising:
a power source operatively coupled to the at least one conductive fiber.

20. A method comprising:
providing a garment comprising an electrifiable fabric comprising non-conductive fibers and conductive fibers, wherein the ratio of non-conductive fibers to fibers is in a range between 2 to 1 and 130 to 1; and
providing electrical power to the electrifiable fabric.
